(19) United States
(12) Plant Patent Application Publication
(10) Pub. No.: US 2004/0025211 P1
(43) Pub. Date: Feb. 5, 2004

(54) Apple tree named 'ROYAL BEAUT'

(76) Inventor: Robert Mohr Zulch, Wakkerstroom (ZA)

Correspondence Address: KNOBBE MARTENS OLSON & BEAR LLP, 2040 MAIN STREET, FOURTEENTH FLOOR, IRVINE, CA, 92614, US

(21) Appl. No.: 10/211,017

(22) Filed: Jul. 31, 2002

(51) Int. Cl. A01H 5/00

(52) U.S. Cl. PLT/162

(57) ABSTRACT

A new and distinct Apple tree variety is disclosed, characterized by a Apple tree which bears an attractive dark red, striped apple which are flavorsome, sweet and crisp, and with good storage quality.

### LATIN NAME OF THE GENUS AND SPECIES

The present invention relates to a new and distinct variety of Apple tree named ‘ROYAL BEAUT’, botanically known as Malus domestica.

### VARIETY DENOMINATION

The new and distinct Apple tree is hereinafter referred to by the name ‘ROYAL BEAUT’.

### BACKGROUND OF THE INVENTION

The present invention relates to a new and distinct variety of apple tree named ‘ROYAL BEAUT’ and more particularly to an apple tree which bears an attractive dark red, striped apples which are flavorsome, sweet and crisp, and with good storage quality. The apples are similar in shape and taste to ‘Royal Gala’, the parent, but the color is much redder and more intense.

### ORIGIN AND ASEXUAL REPRODUCTION OF THE NEW VARIETY

The present invention is a discovered natural mutant Apple tree variety of ‘Royal Gala’. The new tree was discovered and selected by the inventor in Wakkerstroom, Ceres, South Africa, in 1996.

Asexual reproduction of the new cultivar by cleft grafting and bud grafting onto root stock M7/M793 was performed in Ceres and Stellenbosch, South Africa and has shown that the unique features of this cultivar are stable and reproduced true to type on successive generations.

### SUMMARY OF THE INVENTION

The ‘ROYAL BEAUT’ apple tree is characterized principally as to novelty by producing an attractively colored fruit which is good for storage and has very attractive full red coloration with yellow stripes. The present variety is easily distinguishable from the fruit produced by the ‘Royal Gala’ variety that is its parent, in that it has a unique dark red attractive coloring.

A limb mutation on a apple tree of variety Royal Gala exhibited highly colored red striped fruit compared to its parental variety. The new variety has been asexually reproduced and named Royal Beaut. The mutation was noticed because all the apples on a spur were better coloured than the rest of the crop on the tree.

The variety ‘ROYAL BEAUT’ has not been observed under all possible environmental conditions. Testing of this variety was conducted in the Southern Hemisphere. Accordingly, dates and times of year for flowering, fruiting, and the like, will be different for the Northern Hemisphere. The phenotype may vary somewhat with variations in environment such as temperature, daylength, and light intensity, without, however, any variance in genotype.

### BRIEF DESCRIPTION OF THE PHOTOGRAPHS

This new variety of apple tree is illustrated by the accompanying photographic drawings, depicting the plant by the best possible color representation using color photography.

FIG. 1: Close-up of a number of specimens in uniform size, shape and characteristics color and finish.

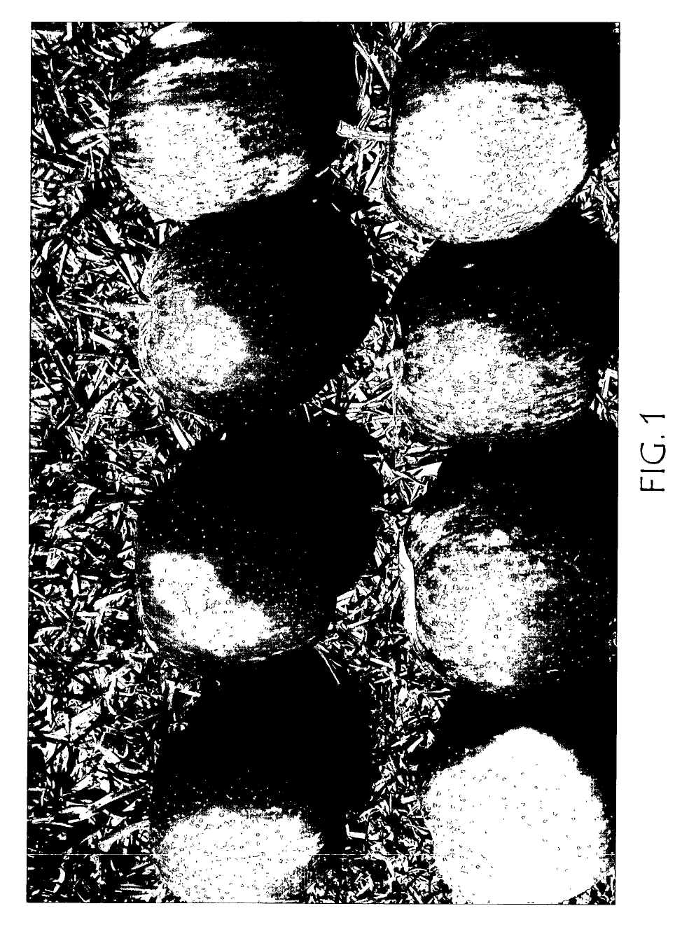

FIG. 2: ‘ROYAL BEAUT’ fruit in situ on a tree of the variety.

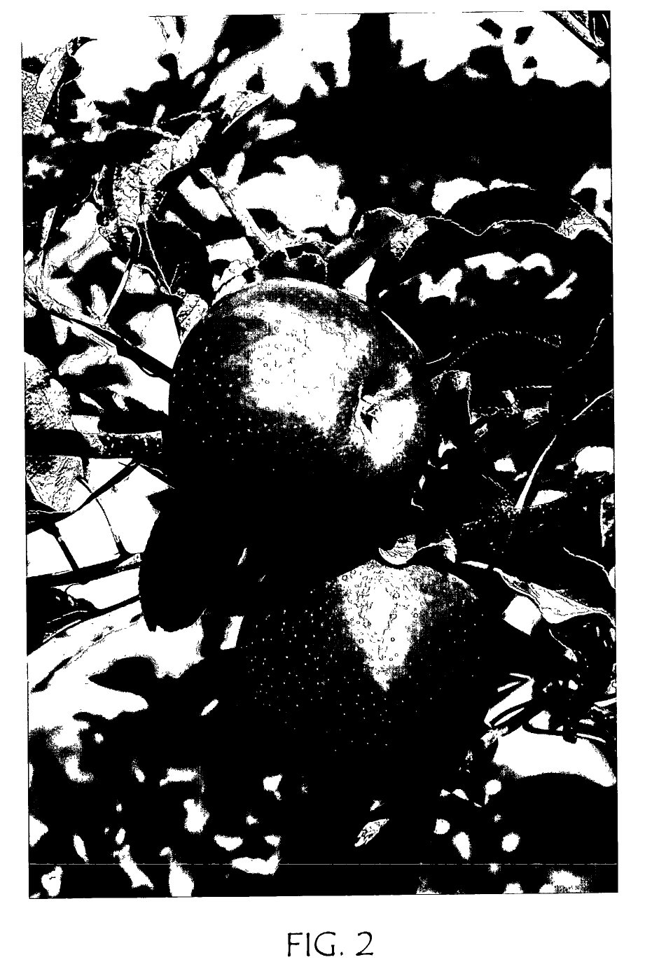

### DETAILED BOTANICAL DESCRIPTION

In the following description, color references are made to The Royal Horticultural Society Colour Chart except where general terms of ordinary dictionary significance are used. The following observations and measurements describe plants grown in Kouebokkeveld, Ceres, South Africa, during the month of August 1995 to July 1996.

Botanical classification.—malus domestica ‘ROYAL BEAUT’.

Commercial classification: Fresh Fruit.

Parentage: Discovered natural mutant of an Apple tree variety, ‘Royal Gala’.

### TREE

Size: The mature trees are approximately 5 m in height, and 2 m in width.

Vigor (as compared to other apple tree varieties): Strong.

Growth rate: Vigorous.

Tree type: Ramified.

Figure of younger, non-bearing trees: Semi-upright.

Figure of older, mature, fruit-bearing trees: Semi-upright.

Trunk:

Bark color.—Light Brown/Green, about 195A (RHS).

Texture of bark.—Smooth.

Dormant one-year-old shoot.—Color of bark: Reddish Brown, about 187A (RHS). Pubescence (on upper half of shoot): Sparse. Shoot thickness: Medium. Angle of shoot with respect to trunk: 45°. Length of internode: Medium. Color of lenticels: White, about 155A (RHS).

Dormant three-year-old shoot.—Color of bark: Light brown/green, about 195A (RHS).

### FOLIAGE

Wood (leaf) buds:

Size.—Large.

Position relative to shoot.—Adpressed.

Time of bud burst.—Beginning of October (in South Africa).

Leaf:

Petiole.—Medium. Average Length: 2.66 cm. Color: Reddish, about 53B (RHS). Diameter: 2 mm. Pubescence: Not Pubescent. Anthocyanin coloration: Slightly (30%).

Stipules.—Relative size (in comparison to other apple trees): Medium to large.

Arrangement.—Alternate.

Attitude in relation to shoot.—Upwards-outwards.

Leaf size.—Mature leaf-width: 92.1 cm. Mature leaf-length: 49.0 cm.

Leaf width.—Medium.

Leaf thickness.—Medium.

Leaf blade.—Ratio of length/width: Medium. Pattern (from main vein): Parallel.

Margin type.—Crenate.

Leaf upper surface.—Color: Dark Green, about 135A (RHS). Young foliage-Upper side: Light green, about 143A (RHS). Mature foliage-Upper side: Green, about 135A (RHS). Leaf texture: Slightly rugose. Glossiness: Weakly glossy. Pubescence: Medium.

Leaf lower surface.—Color: Young foliage-Under side: Green, about 147C (RHS). Mature foliage-Under side: Green, about 147C (RHS). Leaf Texture: Slightly rugose. Glossiness: Glossy. Pubescence: Medium.

### FLOWER

Bloom density: High.

Date of first bloom: Oct. 18, 1995.

Date of full bloom: Oct. 23, 1995.

Flower shape: Perfect.

### FRUIT

General Characteristics:

Fruit size.—Medium.

Fruit weight.—144.9 g.

Fruit diameter.—69.5 mm.

Fruit length.—59.6 mm.

Shape.—Globose conical.

Uniformity.—Uniform shape.

Ribbing.—Absent.

Crowning at calyx end.—Weak.

Length of sepal.—Medium.

Stem cavity:

Depth.—About 13.6 mm.

Breadth.—About 30.0 mm.

Basin (base cavity):

Length.—6.8 mm.

Breadth.—27.9 mm.

Flesh:

Oxidation rate.—Slow.

Juiciness level.—Medium.

Brix (%).—15.0.

Crispness level.—High.

Quality.—High.

Skin:

Ground color.—Yellow, about 20B (RHS).

Color markings/overcolor.—Full red about 46B (RHS) with yellow stripes, about 20B (RHS).

Percentage of overcolor.—>85%.

Fruit lenticels.—Size: Medium.

Skin toughness.—Medium.

Skin smoothness.—Smooth.

Skin greasiness.—Absent.

Propensity to russet.—Sporadic.

Location of russeting when present.—Around stalk.

Tendency to crack.—Absent.

### HARVEST

Average time to bear fruit: 131 days after full bloom.

Harvest period: Beginning to Mid-February (with Royal Gala).

Date of fruit maturity: About February 15^{th}.

Productivity: Good.

Post harvest quality of stored fruits: Good.

Keeping quality: Good.

### MARKET QUALITY OF FRUIT

Main use for: Fresh market.

Appearance: Attractive.

Flavor: Excellent.

Eating quality: High.

### OTHER

Other characteristics:

Chilling requirements.—High, but also does well in warmer areas with rest-breaking agents.

What is claimed is:

1. A new and distinct variety of Apple tree named ‘Royal Beaut’ as herein illustrated and described.
